(19) United States
(12) Patent Application Publication
(10) Pub. No.: US 2018/0168951 A1
(43) Pub. Date: Jun. 21, 2018

(54) FORMULATION

(71) Applicant: COLLTEC LABORATORIES LIMITED, London (GB)

(72) Inventors: David HYLAND, Buckinghamshire (GB); Pupinder Singh GHATORA, Buckinghamshire (GB)

(21) Appl. No.: 15/738,479

(22) PCT Filed: Jun. 23, 2016
(86) PCT No.: PCT/GB2016/051886

§ 371 Date: Dec. 20, 2017

(30) Foreign Application Priority Data
Jun. 26, 2015 (GB) 1511255.0

(51) Int. Cl. A61K 8/11; A61K 8/35; A61K 8/65; A61K 8/73; A61Q 19/08; A23L 33/28

(52) U.S. Cl. CPC A61K 8/11; A61K 8/35; A61K 8/65; A61K 8/735; A61Q 19/08; A23V 2250/5422; A61K 2800/30; A61K 2800/80; A23V 2002/00; A23V 2200/318; A23V 2250/211; A23L 33/28

(57) ABSTRACT

The present invention provides a formulation comprising collagen, wherein the formulation is encapsulated in an enteric capsule and wherein the formulation is substantially free from carriers and free from excipients. Processes for preparing the formulation are also provided.

### FIELD OF INVENTION

The present invention relates to formulations comprising collagen, wherein the formulation is encapsulated in an enteric capsule and is substantially free from carriers and free from excipients, and processes for producing the same.

### BACKGROUND TO THE INVENTION

Reducing levels of natural collagen and hyaluronic acid lie at the heart of the ageing process; which is compounded by UV damage.

Collagen is one of the most abundant group of proteins in the human body and is the major component (80%) of connective tissue. It provides structural support throughout the body where it is found in skin (70% of which is collagen), muscle, ligaments, tendons, cartilage and bones.

A key component of the skin's structure, collagen fibres provide the infrastructure for elastin, which maintains skin elasticity, and for hyaluronic acid to trap moisture. As people age the body's collagen production reduces, which can result in an increase of wrinkles or fine lines, and decreasing smoothness and suppleness of the skin. Indeed, after the age of 25, collagen can be lost at a rate of 1.5% per year.

However, these signs of ageing can be treated or slowed down if total body collagen levels can be increased. Since collagen cannot be absorbed through the skin, people turn to collagen supplementation. Unfortunately, traditional sources of collagen (bovine and porcine) are not considered to be that well absorbed. However, in recent times there has been an advancement in collagen supplementation with the emergence of collagen peptides (especially fish collagen peptides). Studies range from showing increased absorption after ingestion, to other positive efficacious markers of absorption such as the synthesis of new collagen fibres and positive effects on the skin as would be expected by an increase in collagen.

However, not all collagen supplements are effective. Due to the heat sensitive nature of collagen it is generally encapsulated in soft gel capsules, which require little to no heat to be used during the encapsulation process. Collagen and the collagen peptide in hydrolysed collagen is a protein. Once the protein comes into contact with stomach acid the collagen is broken down into its constituent amino acids and will no longer be clinically effective. Liquids, tablets and most capsules are broken down in the stomach in this way and, as a result, insufficient collagen is absorbed to counter the natural depletion of the protein. Conversely, most tablets and capsules if not completely destroyed in the stomach will pass out of the digestive system intact and therefore have no clinical effectiveness.

An object of the present invention is to provide an improved skin care supplement.

### DESCRIPTION

According to a first aspect of the present invention a novel formulation comprising collagen encapsulated in an enteric capsule is provided. Preferably the formulation is substantially free from carriers and substantially free from excipients.

The formulations of the invention can help to replenish lost collagen, thereby reducing the appearance of fine lines and wrinkles and helping to combat the ageing process.

Preferably the formulation is in a form suitable for oral administration to a subject. The subject is preferably a mammal, more preferably a human.

Formulations of the invention are encapsulated in enteric capsules, which are a type of delayed release dosage form. Enteric capsules are resistant to stomach acid, allowing the formulation be released in the small intestine, which provides the largest surface area for absorption of the contents of the capsule. Collagen is known to be especially sensitive to the low pH conditions of the stomach and must survive through gastric and bile acids to reach the gastrointestinal tract to be most effective. In this way the enteric capsules lead to improved absorption of the collagen formulations of the present invention.

The majority of capsules used in the pharmaceutical industry are made from gelatine, i.e., the capsule shell is or comprises gelatine. However, hard gelatine capsules tend to have a relatively high moisture content (typically 13-16% w/w) and are therefore not suited for encapsulating hygroscopic substances, such as collagen. In an embodiment of the invention, the enteric capsule is a hydroxypropyl methylcellulose (HPMC) capsule. HPMC capsule shells used herein have a lower moisture content, generally 10% w/w or less, typically 4-6% w/w compared with gelatine capsules and are therefore more suited for encapsulating hygroscopic substances. Preferred capsule shells of the invention have a moisture content of 10% w/w or less, and more preferred capsules comprise or consist essentially of HPMC.

The collagen may be bovine, porcine or marine collagen and can be hydrolysed collagen, such as collagen peptide. In preferred embodiments of the invention the collagen is marine collagen, preferably hydrolysed marine collagen. Type I, II, III, IV or V collagen can be used in formulations of the invention. Preferably the formulation comprises type I collagen.

In preferred embodiments of the invention the collagen is a collagen peptide, such as a collagen peptide having an average molecular weight of 2,000 Da or less. This can result improved absorption of the collagen in the small intestine.

Formulations of the invention can comprise 50-600 mg collagen, preferably 100-400 mg, more preferably about 266 mg collagen. It will be understood by those skilled in the art that these quantities refer to the amount of collagen in each capsule.

Formulations of the invention may additionally comprise one or more additional active components selected from hyaluronic acid, natural astaxanthin, synthetic astaxanthin, Vitamin A, Vitamin C (ascorbic acid), Vitamin E (tocopherois, tocotrienois), beta carotene, lutein, selenium, silica, alpha lipoic acid, catechins, Coenzyme Q10 (CoQ10), polyphenolic antioxidants (resveratrol, flavonoids), and carotenoids (lycopene, carotenes, lutein). Preferably formulations of the present invention comprise hyaluronic acid and natural astaxanthin.

Astaxanthin is a powerful, naturally occurring carotenoid pigment found in certain marine plants and animals. It is commonly recognised as being one of the most powerful antioxidants found in nature. Unlike many other types of antioxidant astaxanthin can never become a pro-oxidant in the body and will therefore never cause harmful oxidation. Due to its molecular structure astaxanthin is very effective against singlet oxygen. It has a powerful scavenging ability for lipid and free radicals, and effectively breaks peroxide chain reactions. Astaxanthin has been found to improve skin moisture levels, elasticity and smoothness, while reducing wrinkles, freckles and spots.

The structure of astaxanthin is similar to lutein and zeaxanthin, two antioxidants that have been shown to reduce the risk of cataracts. But, while similar, astaxanthin has a stronger antioxidant activity and UV-light protection effect, both of which point to it being an excellent supplement for eye health maintenance. The antioxidant properties of astaxanthin have also been shown to improve heart health, cellular health and the body's immune system as well as having natural anti-inflammatory properties.

Astaxanthin used in formulations of the present invention may be in the form of a powder, preferably a powder formed by micro encapsulation of astaxanthin rich oil. The astaxanthin rich oil can be micro encapsulated in modified starch. This prevents the astaxanthin from oxidising on contact with oxygen molecules in the air, while the oil provides the lipids necessary to maximise absorption.

Formulations of the invention can comprise 0.1-3 mg astaxanthin, preferably 0.8-2 mg, more preferably about 1.3 mg astaxanthin. It will be understood by those skilled in the art that these quantities refer to the amount of astaxanthin in each capsule.

Hyaluronic acid is a glycosaminoglycan found throughout the body of mammals. It is particularly prevalent in connective tissue, the synovial fluid around joints, the eyes, the heart, intervertebral spinal discs and the skin. Hyaluronic acid functions to transport nutrients to cells and remove toxins. It also acts as a cushioning agent, lubricating joints and facilitating water retention in the skin, which is especially important for skin care. Hyaluronic acid can absorb more than one thousand times its weight in water. By increasing the skin's moisture content hyaluronic acid can result in a dramatic improvement in the skin's appearance.

Hyaluronic acid is also heavily involved in promoting and enhancing the skin's collagen matrix. When skin is damaged by free radicals it becomes inflamed, with cells in the dermis producing progressively less hyaluronic acid, which leads to wrinkle formation. Hyaluronic acid levels also naturally decrease with age.

Formulations of the invention may comprise 5-30 mg hyaluronic acid, preferably 10-20 mg, more preferably about 16.6 mg hyaluronic acid. It will be understood by those skilled in the art that these quantities refer to the amount of hyaluronic acid in each capsule.

Hyaluronic acid used in formulations of the invention may have an average molecular weight of 100 kDa or less, preferably 50 kDa or less, more preferably 20 kDa or less. In preferred embodiments of the invention hyaluronic acid has an average molecular weight of about 20 kDa.

In a preferred embodiment of the invention the formulation is substantially free from carriers and free from excipients. Preferably the formulation does not contain any carriers or excipients, or any such components are present at de minimis amounts. Many conventional tablet or capsule supplement formulations contain carriers and excipients in the form of fillers, binders, colourings, disintegrants, diluents and many others. Excipients such as povidone, polyvinylpyrrolidone (PVP), cellulosics and modified starches can constitute up to 85% w/w of a tablet formulation. Some such formulations have been reported to pass through the digestive system unscathed or to get stuck in the digestive system altogether; neither disintegrating, nor passing through. In contrast, the carrier and excipient free formulations of the present invention are free from such problems, leading to better absorption of the active components. In embodiments of the invention the formulation does not contain calcium. More preferably the formulation does not comprise chelated calcium.

In embodiments of the invention collagen, optionally combined with other active ingredients as identified herein, may comprise at least 90% by weight of the contents of the enteric capsule, preferably at least 95% or at least 98% or at least 99% by weight of the contents of the capsule. Preferably collagen, optionally combined with other active ingredients as identified herein, comprises at least 100% by weight of the contents of the enteric capsule.

The formulation may consist essentially of collagen, hyaluronic acid and astaxanthin. In other words, these substances can be encapsulated within the enteric capsule with no other components being present. Thus, collagen, hyaluronic acid and astaxanthin may comprise at least 90% by weight of the contents of the capsule, preferably at least 95% or at least 98% or at least 99% by weight of the contents of the capsule. Preferably, collagen, hyaluronic acid and astaxanthin may comprise 100% by weight of the contents of the capsule.

Formulations of the present invention are particularly suitable for use as a skin care supplement. Preferably the formulations are suitable for oral administration, preferably taken before sleep. For maximal benefits at least one capsule, preferably at least two capsules, more preferably three capsules, should be taken before sleep and at least 1 hour, preferably at least 1.5 hours after eating.

The present invention also provides a process for preparing a formulation as described above, the process comprising: filling enteric capsule shells with collagen and one or more of hyaluronic acid, natural astaxanthin, synthetic astaxanthin, Vitamin A, Vitamin C (ascorbic acid), Vitamin E (tocopherois, tocotrienois), beta carotene, lutein, selenium, silica, alpha lipoic acid, catechins, Coenzyme Q10 (CoQ10), polyphenolic antioxidants (resveratrol, flavonoids), and carotenoids (lycopene, carotenes, lutein), with no other components being included; sealing the capsule shells; and cleaning the sealed capsules. The process is preferably carried out under positive pressure to ensure that hygroscopic components of the formulation are not exposed to moisture.

### Example 1: Making and Encapsulating the Formulation

Capsule filling and packaging is undertaken by contract manufacture.

The capsules are filled and bottled in an ISO Class 9 Pharmaceutical clean room, which means the air and temperature levels are automatically controlled with 20 complete air changes every hour. The entire production process from unpacking the raw materials to bottling the capsules happens inside the clean room to maintain product quality.

The steps undertaken in the clean room are:
1. Raw material testing.
2. Raw material mixing—sourced active ingredients are mixed in the required volume for the formula without any additions.
3. Capsule filling—the sourced capsule shells are then filled with the formula powder, using a Trufil™ approach, and sealed.
4. Capsule cleaning—the capsules are tumble cleaned to remove any excess powder.
5. Capsule inspection.
6. Bottle filling—the capsules are then bottled and the bottles sealed with neck seals.

Final packaging and final quality control then takes place outside the clean room environment.

Once filled, each capsule contains 1.3 mg astaxanthin, 16.7 mg hyaluronic acid and 266 mg collagen.

The capsules do not contain any ingredients other than the active ingredients and thereby avoid problems associated with the presence of fillers, binders, excipients, colourings, disintegrates and diluents etc., which can be detrimental to absorption of the active ingredients.

Capsule Shells

Capsule shells (DRcaps®) are sourced from Capsugel®.

Protecting active ingredients which are sensitive to the active environment of the stomach can be difficult. Often a delivery system solution is used that requires significant investment and lengthy processes after encapsulation. The sourced capsules are an acid-resistant alternative that provides protection. These capsules, made from hydroxypropyl methycellulose (HPMC), are resistant to a low pH environment such as that found in the stomach.

The capsules also have a low moisture content, making them an ideal oral dosage container for moisture sensitive or hygroscopic food supplement ingredients; collagen peptides are highly hygroscopic.

Marine Collagen Peptide

Enzymatically hydrolysed marine collagen peptide with an average molecular weight of 2,000 Da (Peptan® F2000HD) is sourced from Rousselot®.

To ensure maximum absorption collagen peptides with an average molecular weight of 2,000 Da are used; molecules of this weight have been shown to be readily absorbed by the small intestine.

Raw materials for the collagen peptide come exclusively from fish from facilities registered by the European Union and deemed fit for human consumption by official veterinarian post mortem inspection. The manufacturing process consists of the following steps: extraction with hot water, filtration, concentration, enzymatic hydrolysis, sterilisation at 138° C., and spray drying.

Astaxanthin

Encapsulated natural astaxanthin (AstaReal® EL25) is sourced from AstaReal®.

There are two major problems in using astaxanthin in conjunction with a collagen peptide. These are:

1. Astaxanthin is exceptionally unstable when subjected to oxygen. Because it is such a strong antioxidant, the astaxanthin molecule will begin to bond with oxygen molecules in the air. Once it oxidizes, it will break down into a degradation product called “astacene” that has no benefit. Extreme care must therefore be taken when handling astaxanthin to be sure that it does not oxidize.

2. It is also highly recommended that astaxanthin is taken with meals, preferably with a meal that has some fat content. This is similar to other carotenoids, all of which are fat soluble. These lipophilic (“fat-loving”) nutrients, taken in the absence of fats, are poorly absorbed; when taken with fats, the absorption is maximized. Collagen peptide absorption, on the other hand, is maximised if it is taken when the stomach is relatively empty.

These issues have been overcome by using a micro encapsulated astaxanthin rich oil with a significant lipid content; this approach both protects the astaxanthin molecule against oxidisation and provides the fat content required to maximise absorption.

The powder is manufactured by micro encapsulation of astaxanthin rich oil in modified starch. The astaxanthin is produced by non-solvent supercritical carbon dioxide extraction of crushed and spray dried aplanospores of the green microalga Haematococcus pluvialis.

Hyaluronic Acid

Pharmaceutical quality hyaluronic acid with an average molecular weight of 20 kDa is sourced from Ubichem.

The hyaluronic acid is manufactured by bacterial fermentation with a natural non-GMO strain, under GMP conditions and is of non-animal origin.

### Example 2

In 2008 an independent trial of Peptan® F2000 Collagen Peptide in a non-enteric vegetable cellulose capsule was conducted by a third party, Ayton Online Research Limited, with a panel of 100 participants consisting of females between the ages of 35 and 65.

The trial involved each participant taking the recommended dose of 3 capsules nightly for a period of three months. Each participant was asked to complete a questionnaire before commencing the trial; at the end of months 1, 2, and 3; and on completion of the trial.

Peptan F2000 is a marine collagen peptide with an average molecular weight of 2,000 Da manufactured from the skin of Tilapia by enzymatic hydrolysis.

The questions and results from end of trial questionnaires were as follows:

1. How do you feel the product has improved the plumpness of your skin as compared to the start of the trial? Select score between 1 and 9 (1=not at all, 9=a lot)

Over 89% of the participants indicated that they had noticed some improvement in the plumpness of their skin, with 72% scoring 5 or more against this question.

2. How do you feel the product has improved the firmness of your skin as compared to the start of the trial? Select score between 1 and 9 (1=not at all, 9=a lot)

Over 86% of the participants indicated that they had noticed some improvement in the firmness of their skin, with 73% scoring 5 or more against this question.

3. How do you feel the product has improved the elasticity of your skin as compared to the start of the trial? Select score between 1 and 9 (1=not at all, 9=a lot)

Over 85% of the participants indicated that they had noticed some improvement in the elasticity of their skin, with 67% scoring 5 or more against this question.

4. How effective was the product at smoothing the skin? Select score between 1 and 9 (1=poor, 9=good)

Over 89% of the participants indicated that they had noticed some smoothing of their skin, with 68% scoring 5 or more against this question.

5. How effective was the product at repairing the skin? Select score between 1 and 9 (1=poor, 9=good)

Over 88% of the participants indicated that the product improved skin repair, with 67% scoring 5 or more against this question.

6. How effective was the product at strengthening the skin? Select score between 1 and 9 (1=poor, 9=good)

Over 88% of the participants indicated that they had noticed some strengthening of their skin, with 69% scoring 5 or more against this question.

7. How effective was the product at brightening the skin? Select score between 1 and 9 (1=poor, 9=good)

Over 89% of the participants indicated that they had noticed some brightening of their skin, with 70% scoring 5 or more against this question.

8. How do you feel the product has improved the appearance of your skin tone as compared to before the trial? Select score between 1 and 9 (1=not at all, 9=a lot)

Over 86% of the participants indicated that they had noticed some improvement in skin tone, with 71% scoring 5 or more against this question.

9. How do you feel the product has improved the youthfulness of your skin as compared to the start of the trial? Select score between 1 and 9 (1=not at all, 9=a lot)

Over 85% of the participants indicated that they had noticed some improvement the youthfulness of their skin, with 66% scoring 5 or more against this question.

10. How effective was the product at strengthening your nails? Select score between 1 and 9 (1=poor, 9=good)

Over 86% of the participants indicated that they had noticed some strengthening of their nails, with 68% scoring 5 or more against this question.

11. How effective was the product at strengthening your hair? Select score between 1 and 9 (1=poor, 9=good)

Over 87% of the participants indicated that they had noticed some strengthening of their hair, with 66% scoring 5 or more against this question.

12. How effective was the product at improving the thickness of your hair? Select score between 1 and 9 (1=poor, 9=good)

Over 84% of the participants indicated that they had noticed some thickening of their hair, with 62% scoring 5 or more against this question.

13. How do you feel the product has improved your sleep patterns as compared to before the trial? Select score between 1 and 9 (1=not at all, 9=a lot)

Over 77% of the participants indicated that they had noticed some improvement in their sleep patterns, with 62% scoring 5 or more against this question.

14. How effective was the product at improving your body's vitality as compared to the start of the trial? Select score between 1 and 9 (1=not at all, 9=a lot)

Over 80% of the participants indicated that they had noticed some improvement in their vitality, with 58% scoring 5 or more against this question.

15. How would you rate the product overall? Select score between 1 and 9 (1=poor, 9=good)

The average rating for the product overall was 5.56. However, 63% of the participants scored the product at 5 or more.

Unsolicited comments made by participants on completion of the trial included:
Wow this product really works my thin hair is now so thick people are now asking me if I'm wearing a hair piece it looks like I've got so much and lots of new hair growth I can't believe it. My nails are super strong and long which I've not had since my early teens. I'm quite lucky I didn't have many lines but my skin does feel so much tighter. I have fibromyalgia and my sleep was far more restful than before this treatment and my general wellbeing was much better. I want to purchase these I've found something that helps me.
This is a fantastic product which gives all round results. The extra sleep I've been getting has probably helped me look and feel better, too. Thrilled with the results in the short, medium and long term.
Fantastic product, great for clearing up skin problem and helping with sleep, but I found myself being really tired in the mornings, this was reduced however when I went down to 2 at night.
No noticeable effect on sleep patterns, little effect on nails, but a good result with skin tone and wrinkles. Would buy this product.
I still am amazed slightly that something I take has made such a difference to my skin as well as everything else.
Brilliant.
I wish I knew what this was so that I could buy it . . . is there any way that the company could contact me??? But it is a very good product . . . I look fabulous and friends have commented.
An excellent product that was really effective.
My skin looks a lot more radiant and shiny. I look younger than I ever did and I feel fantastic. Many people have mentioned how well I am looking and how perfect my skin is. I really have noticed the changes. This product is excellent. I would recommend it to anyone who wants to look younger and more radiant. Thanks for letting me take part.
I feel better for taking the tablets—mainly because it seems to have helped my joints feel more supple and stopped them aching.
It worked fantastically overall my body feels much better. My limbs are a lot better and the move ability feels much better. My nails are shinier and stronger. My hair is thicker and shinier. My face is the most improvement it looks so much younger and my skin suppler and smoother.
Very easy to swallow, hair looks a bit thicker, my sleep has improved.
My nails are so long and in excellent condition.
Seems to be making a good difference through continued use!
It has improved my nails, hair and skin.
The fact that is has helped my joints.
Thickened my hair.
The way that they improved my wellbeing.
The difference in my nails.
My hair felt better.
How great my skin looks and feels after use.
My improved sleep patterns.
The positive effect it had on fine lines.
How great my skin looks and feels after use.
This has made my skin tone more even and smoother.
Loved the fact that my spots all cleared up.
Easy to take, results on skin noticeable within days.
It was good at strengthening my hair and nails. It made my hair look much healthier.
Very good all round product.
Hair looks shiny, sleep has improved.
I will keep buying this type of collagen I do feel better.
Very impressed with how it improved over time!
The product has improved my hair and nails greatly.
Improvements in all areas—mostly nails are a lot stronger, well worth continuing with—thank you. Would recommend.
Great product. I'm pretty sure it has helped minimise my pores too.
This dietary supplement gave me a feeling of well-being.
Good product with good benefits.
Overall, my skin looks and feels plumper.
It was easy to remember to take as just one dose a day.
Easy to administer and as stated great improvement in strength of nails.
The effect on my skin.
Brightened skin.
I did sleep better, and my nails grew quicker and were stronger.
My plumper skin and lips.
I felt that the product was aiding my health and attitude.
The way it's made me look better, I finally have a lovely complexion.
The way it makes me think my skin looks better.
Everything fantastic results.
Easy to take, no fuss and very effective.
It made me look glowing and healthy.
It was so much easier to take a capsule than spend hours applying creams.
The overall benefits.
The improvement to hair and nails.
The way my skin felt.
Effects were amazing.

1. A formulation comprising collagen, wherein the formulation is encapsulated in an enteric capsule and wherein the formulation is substantially free from carriers and free from excipients.

2. The formulation according to claim 1, wherein the collagen is a collagen peptide having an average molecular weight of 2,000 Da or less.

3. The formulation according to claim 1, wherein the formulation comprises one or more additional active components selected from hyaluronic acid, natural astaxanthin, synthetic astaxanthin, Vitamin A, Vitamin C (ascorbic acid), Vitamin E (tocopherois, tocotrienois), beta carotene, lutein, selenium, silica, alpha lipoic acid, catechins, CoQ10, polyphenolic antioxidants (resveratrol, flavonoids), and carotenoids (lycopene, carotenes, lutein).

4. The formulation according to claim 1, wherein the formulation consists essentially of collagen, hyaluronic acid and astaxanthin.

5. The formulation according to claim 4, wherein the formulation comprises 50-600 mg collagen.

6. The formulation according to claim 4, wherein the formulation comprises 5-30 mg hyaluronic acid.

7. The formulation according to claim 4, wherein the formulation comprises 0.1-3 mg astaxanthin.

8. The formulation according to claim 1, for use as a skin care supplement.

9. A process for preparing the formulation according to claim 1, the process comprising:
filling enteric capsule shells with collagen and one or more of hyaluronic acid, natural astaxanthin, synthetic astaxanthin, Vitamin A, Vitamin C (ascorbic acid), Vitamin E (tocopherois, tocotrienois), beta carotene, lutein, selenium, silica, alpha lipoic acid, catechins, CoQ10, polyphenolic antioxidants (resveratrol, flavonoids), and carotenoids (lycopene, carotenes, lutein), with no other components being included;
sealing the capsule shells; and
cleaning the sealed capsules.
